(19) United States
(12) Patent Application Publication
(10) Pub. No.: US 2022/0273521 A1
(43) Pub. Date: Sep. 1, 2022

(54) METHODS OF ACCURATELY PLACING A GASTRIC TUBE IN A SUBJECT

(71) Applicant: Ahmad Oussama Rifai, Lynn Haven, FL (US)

(72) Inventor: Ahmad Oussama Rifai, Lynn Haven, FL (US)

(21) Appl. No.: 17/014,219

(22) Filed: Sep. 8, 2020

Related U.S. Application Data
(60) Provisional application No. 62/896,103, filed on Sep. 5, 2019.

(51) Int. Cl. A61J 15/00; A61B 1/05; A61B 1/267; A61M 16/04

(52) U.S. Cl. CPC A61J 15/0003; A61B 1/05; A61B 1/267; A61M 16/0463

(57) ABSTRACT

The present disclosure provides methods of accurately placing a gastric tube, such as an orogastric tube or a nasogastric tube, in an esophageal orifice of a subject using an airway visualization system.

### PRIORITY CLAIM

This application claims priority to U.S. Provisional Patent Application Ser. No. 62/896,103, filed Sep. 5, 2019, the entire contents of which are incorporated herein by reference and relied upon.

### BACKGROUND

Current methods of accurately placing a gastric tube (e.g., an orogastric tube or a nasogastric tube) in a subject in need thereof typically require radiography or other tests to verify safe and accurate insertion. Often, a subject in need of gastric tube intervention has already been endotracheally intubated, rendering gastric tube placement more prone to misplacement and even harm to the subject. A need persists for methods of proactively, quickly and accurately placing a gastric tube in a subject in need thereof.

### SUMMARY

The present disclosure provides methods of using an airway visualization system (e.g., a fiber optic video-based system such as GlideScope) to accurately insert a gastric tube into a subject in need thereof.

In one embodiment, the present disclosure provides a method of accurately placing an orogastric tube in a subject in need thereof, the method comprising: inserting a distal portion of an airway visualization system into the hypopharynx of the subject; and inserting an orogastric tube into the superior esophageal orifice of the subject.

In another embodiment, the present disclosure provides a method of accurately inserting a nasogastric tube in a subject in need thereof, the method comprising: inserting a distal portion of an airway visualization system into the hypopharynx of the subject; inserting a distal end of a nasogastric tube into a nostril of the subject; advancing the nasogastric tube until the distal end is proximal to the superior esophageal orifice; and advancing the distal end of the nasogastric tube into the superior esophageal orifice of the subject.

In another embodiment, the present disclosure provides a method of treating a subject in need thereof, the method comprising: inserting a distal portion of an airway visualization system into the hypopharynx of the subject; inserting an endotracheal tube into the laryngeal orifice through the vocal cords into the trachea of the subject; inserting the distal portion of the airway visualization system into the hypopharynx of the subject; inserting a gastric tube into the superior esophageal orifice of the subject; and thereafter removing the distal portion of the airway visualization system from the subject.

These and other embodiments are described in greater detail below.

### BRIEF DESCRIPTION OF THE DRAWINGS

FIG. 1 shows a screen capture from an airway visualization system of a hypopharynx after placing an orogastric tube in the esophagus of a subject previously intubated with an endotracheal tube, according to one embodiment of the present disclosure.

FIG. 2 shows a survey used to collect data from clinicians performing a method of placing an orogastric tube in the esophagus of a subject previously intubated, according to one embodiment of the present disclosure.

The figures depict various embodiments of this disclosure for purposes of illustration only. One skilled in the art will readily recognize from the following discussion that alternative embodiments of the structures and methods illustrated herein may be employed without departing from the principles of embodiments described herein.

### DETAILED DESCRIPTION

Gastric tube placement is required after a subject is intubated in order to deliver nutrients, medications and/or oral contrast dyes, or to decompress stomach fluid, blood or air. Frequently, subjects requiring gastric tube placement have respiratory failure (e.g., acute respiratory failure) that requires intubation and gastric tube placement.

Placement of gastric tubes is commonly performed “blind”—that is, the clinician does not (and often cannot) see the hypopharynx of the subject while the gastric tube is advanced into the superior esophageal orifice. The traditional “blind” method carries risks of incorrect gastric tube placement; consequences of inaccurate gastric tube placement can be severe—ranging from localized bleeding, placement into the lung, or albeit rare, into the cranium. Usually, there is a delay between time of endotracheal intubation and time of placing of a gastric tube; the technologies and methods of the present disclosure improves accuracy, reduce complications, side effects, and patient stress by reducing that time delay.

Simple methods to verify accurate gastric tube placement are generally insufficient. For example, epigastric and left upper quadrant auscultation alone cannot positively verify accurate gastric tube placement. Gastric tube aspirate, pH testing, and carbon dioxide monitoring are insufficiently accurate when employed individually. Confirming gastric tube placement by abdominal radiograph, while considered to be the gold standard, involves significantly higher costs than the above alternatives, and also increases radiation exposure risks for the subject while delaying initiation of therapy.

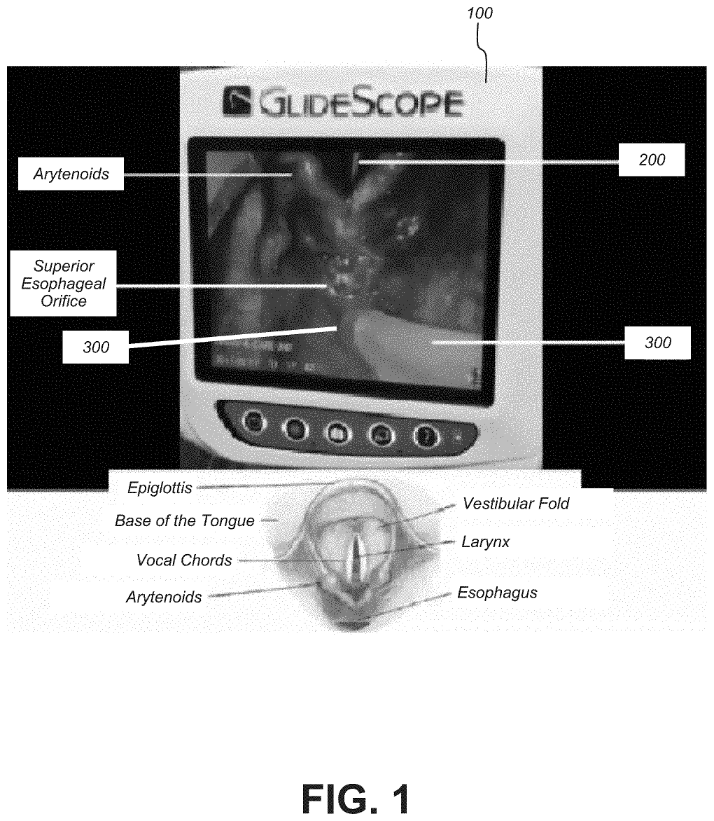

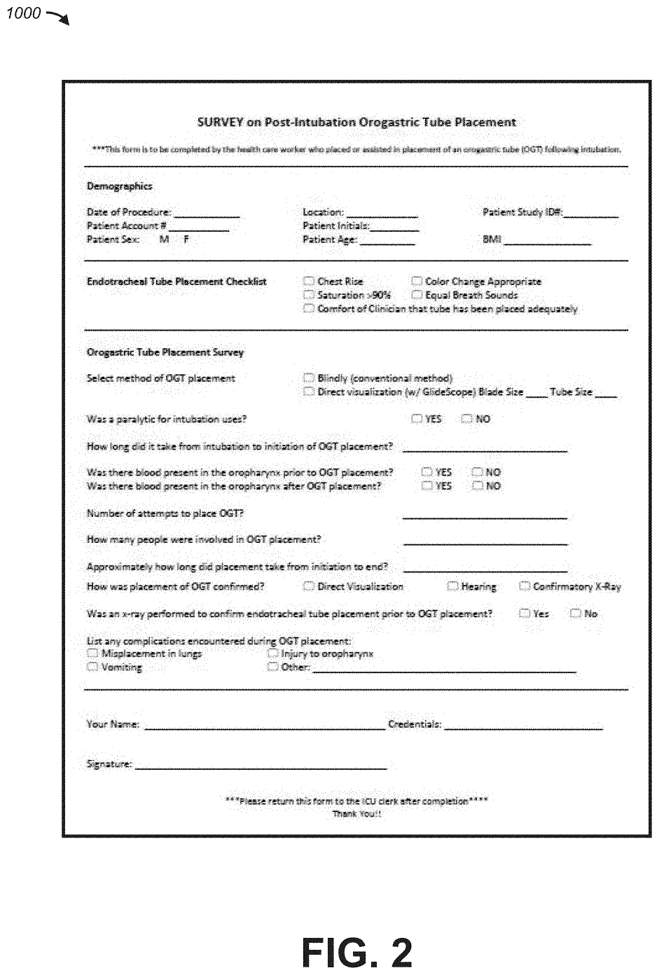

Referring generally to FIGS. 1-2, the present disclosure provides methods of using an airway visualization system 100 to accurately place a gastric tube 300 (e.g., an orogastric tube or a nasogastric tube) in the superior esophageal orifice of a subject, for example in the esophagus of a subject that has previously been intubated with an endotracheal tube 200. Such methods substantially improve accurate placement of a gastric tube 300 in a subject's superior esophageal orifice, while avoiding the need for obtaining a confirmatory abdominal radiograph.

In general, methods consistent with the present disclosure comprise visualizing the hypopharynx of a subject using an airway visualization system 100, and inserting the distal end 310 of a gastric tube 300 into the superior esophageal orifice of the subject.

The gastric tube 300 may be any suitable orogastric tube that is approved for orogastric placement (e.g., Levin Tube, part no. 0044120, Bard Medical Division, Covington, USA), or any suitable nasogastric tube that is approved for nasogastric placement (e.g., Feeding Tube, part no. 0036420, Bard Medical Division, Covington, USA), such as a dobhoff tube (e.g., Kangaroo Feeding Tube part no. 8884710867, Cardinal Health).

The airway visualization system 100 may be any suitable image-based (e.g., video-based) visualization system, such as a GlideScope system (e.g., GlideScope Core, Verathon, Inc., Bothell, Wash., USA). In general, the airway visualization system 100 includes a video monitor in communication with an image capture device (e.g., a camera) that is associated with a bronchoscope or laryngoscope. Visualization of the hypopharynx of the subject is achieved by inserting the bronchoscope or laryngoscope into the mouth or the nose, if the device is approved for nasal insertion, of the subject and viewing images from the image capture device on the video monitor.

If the subject has already been intubated, for example using an airway visualization system 100, placement of the gastric tube 300 into the superior esophageal orifice of the intubated subject may be accomplished by sweeping the endotracheal tube 200 under the blade of the laryngoscope posterior to the endotracheal tube, visualizing the superior esophageal orifice with the airway visualization system 100, and thereafter advancing the distal end 310 of the gastric tube 300 into the superior esophageal orifice. After successful placement of the gastric tube 300 into the esophagus, the airway visualization system 100 may be removed from the subject's mouth.

Intubation and gastric tube 300 placement may be conveniently accomplished by first visualizing the subject's hypopharynx using an airway visualization system 100, inserting an endotracheal tube 200, thereafter visualizing the subject's superior esophageal orifice with the airway visualization system 100, and then placing the gastric tube 300 in the subject's esophagus. In such embodiments, the method is proactive, rather than the current standard reactive.

The current standard practice of intubation (optionally with the aid of an airway visualization system 100), followed by blind placement of a gastric tube 300 coupled with functional and/or radiographic confirmation of accurate gastric tube 300 placement.

In some embodiments, confirmation of accurate placement of the gastric tube 300 may be accomplished by abdominal or chest radiography (e.g., x-ray). If the subject is also intubated, a single radiograph may be obtained after intubation and gastric tube 300 placement to confirm proper placement of both the endotracheal tube 200 and the gastric tube 300.

However, radiography to confirm proper placement of the gastric tube 300 may not be necessary when the gastric tube 300 is placed by a method consistent with the present disclosure. In some embodiments, therefore, the method does not include a step of obtaining an abdominal radiograph (e.g., by x-ray) to confirm proper placement of the gastric tube 300.

In some embodiments, confirmation of proper gastric tube 300 placement may be accomplished functionally, for example by injecting air into the gastric tube 300 after insertion into the subject's esophagus and subsequent advancement of the distal end 310 into the subject's stomach, and then auscultating the subject's epigastric area.

In some embodiments, a sedative and/or paralytic agent is administered to the subject before insertion of the gastric tube 300 into the subject's esophagus. For example and without limitation, the subject may be administered, or may have been administered, a suitable dose of etomidate, a benzodiazepine (e.g., midazolam, lorazepam, or diazepam), ketamine, propofol, ketofol (i.e., a combination of ketamine and propofol), thiopental, methohexital, succinyl choline or any paralytic agent or a clinically useful combination of two or more of the foregoing.

In some embodiments, a sedative or a paralytic agent are not administered to the subject immediately before placement of the gastric tube 300 into the subject's esophagus. For example, in embodiments wherein placement of the gastric tube 300 is performed subsequent to placement of an endotracheal tube 200 into the subject's trachea and wherein the subject is still suitably sedated and/or paralyzed from administration of a sedative and/or a paralytic agent, respectively, to perform the intubation procedure, administration of additional sedative or paralytic agent is not necessary to successfully place the gastric tube 300 in the subject's esophagus.

In some embodiments, such as when the subject was intubated more than about 10 minutes prior, the subject may be administered a sedative and/or a paralytic agent immediately before placement of the gastric tube 300 into the subject's esophagus. For example, an intubated subject may be administered etomidate, a benzodiazepine (e.g., midazolam, lorazepam, or diazepam), ketamine, propofol, ketofol (i.e., a combination of ketamine and propofol), thiopental, methohexital, succinyl choline or any paralytic agent or a clinically useful combination of two or more of the foregoing, immediately before visualizing the superior esophageal orifice using an airway visualization system 100 and subsequently inserting the gastric tube 300 into the esophagus of the subject.

Insertion of the gastric tube 300 into the esophagus of an intubated subject may include sweeping the endotracheal tube 200 under a blade associated with the distal portion of the airway visualization system 100 (e.g., a spectrum blade or a stat associated with the bronchoscope or the laryngoscope) before the step of inserting the gastric tube 300 into the subject's esophagus.

Insertion of the gastric tube 300 into the subject's esophagus includes advancing the distal end 310 of the gastric tube 300 posterior to the larynx at the subject's midline, and into the superior esophageal orifice.

In embodiments wherein the gastric tube 300 is a nasogastric tube, the distal end 310 of the nasogastric tube is first advanced into the subject's nostril and to the hypopharynx (e.g., until the distal end 310 is proximal to the superior esophageal orifice). Insertion of the distal end 310 of the nasogastric tube into the subject's esophagus then proceeds by visualizing the superior esophageal orifice with the airway visualization system 100, sweeping the endotracheal tube (if present) under the blade of the distal portion of the airway visual system 100, and then advancing the distal end 310 of the nasogastric tube into the superior esophageal orifice of the subject.

After the distal end 310 of the gastric tube 300 has been advanced into the subject's esophagus, the airway visualization system 100 is removed from the subject's mouth. Optionally, the airway visualization system 100 can be utilized to confirm a lack of trauma to tissues near the subject's hypopharynx and/or superior esophageal orifice before removing the airway visualization system 100 from the subject's mouth. The distal end 310 of the gastric tube 300 can be advanced to the subject's stomach before or after removal of the airway visualization system 100 from the subject's mouth.

In some embodiments, the present disclosure provides a method of accurately placing an orogastric tube in a subject in need thereof, the method comprising: inserting a distal portion of an airway visualization system into the hypopharynx of the subject; and inserting an orogastric tube into the superior esophageal orifice of the subject. In some embodiments, the airway visualization system is a fiber optic video-based system. In some embodiments, the fiber optic video-based system is a GlideScope. In some embodiments, the method does not include verifying proper orogastric tube placement by radiography. In some embodiments, the method further comprises administering a paralytic agent to the subject before the step of inserting the orogastric tube. In some embodiments, the subject has an endotracheal tube installed in a trachea of the subject. In some embodiments, the method further comprises sweeping the endotracheal tube under a blade associated with the distal portion of the airway visualization system before the step of inserting the orogastric tube. In some embodiments, the method further comprises removing the distal portion of the airway visualization system from the hypopharynx of the subject after the step of inserting the orogastric tube. In some embodiments, the step of inserting the orogastric tube comprises: exposing the superior esophageal orifice with the airway visualization system; and thereafter advancing a distal end of the orogastric tube posterior to the larynx at the midline of the subject's hypopharynx and into the superior esophageal orifice. In some embodiments, the method further comprises injecting air into the orogastric tube after the step of inserting the orogastric tube, and auscultating an epigastric area of the subject to confirm placement of a distal end of the orogastric tube in a stomach of the subject. In some embodiments, the method is associated with a substantially reduced risk of improper orogastric tube placement.

In some embodiments, the present disclosure provides a method of accurately inserting a nasogastric tube in a subject in need thereof, the method comprising: inserting a distal portion of an airway visualization system into a hypopharynx of the subject; inserting a distal end of a nasogastric tube into a nostril of the subject; advancing the nasogastric tube until the distal end is proximal to the superior esophageal orifice; and advancing the distal end of the nasogastric tube into the superior esophageal orifice of the subject. In some embodiments, the airway visualization system is a fiber optic video-based system. In some embodiments, the fiber optic video-based system is a GlideScope. In some embodiments, the method does not include verifying proper nasogastric tube placement by radiography. In some embodiments, method further comprises administering a paralytic agent to the subject before the step of inserting the nasogastric tube. In some embodiments, the subject has an endotracheal tube inserted in a trachea of the subject. In some embodiments, the method further comprises sweeping the endotracheal tube under a blade associated with the distal portion of the airway visualization system before the step of inserting the nasogastric tube. In some embodiments, the method further comprises removing the distal portion of the airway visualization system from the hypopharynx of the subject after the step of inserting the nasogastric tube. In some embodiments, the step of inserting the nasogastric tube comprises: exposing the superior esophageal orifice with the airway visualization system; and thereafter advancing a distal end of the nasogastric tube posterior to the larynx at a midline of the subject and into the superior esophageal orifice. In some embodiments, the method further comprises injecting air into the nasogastric tube after the step of inserting the nasogastric tube, and auscultating an epigastric area of the subject to confirm placement of a distal end of the nasogastric tube in a stomach of the subject. In some embodiments, the method is associated with a substantially reduced risk of improper nasogastric tube placement.

In some embodiments, the present disclosure provides a method of treating a subject in need thereof, the method comprising: inserting a distal portion of an airway visualization system into the hypopharynx of the subject and visualizing the vocal cords; inserting an endotracheal tube into the larynx and through the vocal cords of the subject; inserting the distal portion of the airway visualization system into the hypopharynx of the subject; inserting a gastric tube into the superior esophageal orifice of the subject; and thereafter removing the distal portion of the airway visualization system from the subject. In some embodiments, the method does not include removing the distal portion of the airway visualization system from an oral cavity of the subject between the steps of inserting the endotracheal tube and the step of inserting the gastric tube. In other embodiments, the method comprises removing the distal portion of the airway visualization system from the oral cavity of the subject after the step of inserting the endotracheal tube and reinserting the distal portion of the airway visualization system into the oral cavity of the subject and thereafter sweeping the endotracheal tube under the blade of the airway visualization system before the step of inserting the gastric tube. In some embodiments, the airway visualization system is a fiber optic video-based system. In some embodiments, the fiber optic video-based system is a GlideScope. In some embodiments, the method does not include verifying proper gastric tube placement by radiography. In some embodiments, the method further comprises administering a paralytic agent to the subject before the step of inserting the gastric tube. In some embodiments, the step of inserting the distal portion of the airway visualization system comprises sweeping the endotracheal tube under a blade associated with the distal portion of the airway visualization system before the step of inserting the gastric tube. In some embodiments, the method further comprises removing the distal portion of the airway visualization system from the hypopharynx of the subject after the step of inserting the gastric tube. In some embodiments, the gastric tube is an orogastric tube. In some embodiments, the gastric tube is a nasogastric tube. In some embodiments, the step of inserting the nasogastric tube comprises: exposing the superior esophageal orifice with the airway visualization system; and thereafter advancing a distal end of the nasogastric tube posterior to the larynx at the midline of the subject's hypopharynx and into the superior esophageal orifice. In some embodiments, the method further comprises injecting air into the gastric tube after the step of inserting the gastric tube, and auscultating an epigastric area of the subject to confirm placement of a distal end of the gastric tube in a stomach of the subject. In some embodiments, the method is associated with a substantially reduced risk of inaccurate gastric tube placement. In some embodiments, the subject has respiratory failure. In some embodiments, the subject has acute respiratory failure. In some embodiments, the subject has received a sedative before the step of inserting the distal portion of the airway visualization system into the hypopharynx of the subject.

### EXAMPLE

An investigational study was conducted to determine safety of using an airway visualization system to accurately insert an orogastric tube in sedated patients that were already intubated.

### Subject Recruitment and Study Protocol

Sixteen previously endotracheally intubated subjects, of which 7 were male and 9 were female, collectively having an age range of 47-83 years, and a BMI range of 20.7-63.6 were recruited for an investigational study if all of the following four inclusion criteria were met immediately after endotracheal intubation:
1) Oxygenation saturation levels above 90%;
2) Observation and palpation of chest rise with auscultation in the bilateral axillary areas with the finding of equal breath sounds present;
3) Appropriate color change of the colorimetric end-tidal carbon dioxide detection device; and
4) Absence of sounds with auscultation and absence of movement and distention in the epigastric area.

Subjects who were not already sedated received etomidate followed by succinylcholine for sedation. A GlideScope (Verathon Inc., Bothell, Wash.) equipped with a blade size 3 or 4 was inserted into the oral cavity of the subject, and the endotracheal tube was swept under the blade. The esophageal orifice was then exposed and an orogastric tube (Bard Medical Division, Covington, USA) was advanced posterior to the larynx at the midline.

Spontaneous swallowing movements were sometimes observed in patients who were not paralyzed before intubation or in those recovering from a paralytic agent. The orogastric tube was advanced to the predetermined length or until gastric contents appeared in the orogastric tube. Air was then injected into the orogastric tube while auscultating the epigastric area to confirm that the tube was located in the stomach.

A chest radiograph for endotracheal tube placement was also used to evaluate the orogastric tube. This single chest radiograph was used to confirm both endotracheal tube placement and orogastric tube position. After intubation and orogastric tube placement using method disclosed herein, the provider completed the form depicted in FIG. 2.

### Results

We observed that the time between completion of oral endotracheal intubation and initiation of orogastric tube placement was less than 5 minutes, except in Patient 12, in whom the procedure took 24 hours (clearance from the gastrointestinal services was required due to esophageal varices), and Patient 14, who required a central line before orogastric tube placement because of hemodynamic instability and the need for a large IV access.

Patient 3, Patient 8, and Patient 12 had blood in the oropharynx after orogastric tube placement, but they each also had blood prior to orogastric tube placement. There was no trauma leading to bleeding in our patients. One patient, Patient 1, needed both the physician and physician assistant to place the orogastric tube, whereas the 15 subsequent subjects had the orogastric tube successfully placed by only one provider, namely the provider performing the endotracheal intubation. Patient 1 was intubated by a physician assistant; however, when the attempt to place an orogastric tube failed, the physician was required to place the orogastric tube in this subject. This failed attempt in the first subject may be associated with the learning curve of the procedure tested in this study.

There was no need for a separate chest radiograph to confirm the orogastric tube placement, except for Patient 12, who had already received chest radiography 24 hours prior to confirm the endotracheal intubation. Only one chest radiograph was required for confirmation of the correct position of both endotracheal tube and orogastric tube. No additional complications were observed. Full study data appears in Table 1A (Patients #1-8) and Table 1B (Patients #9-16) below.

TABLE 1A
Study Results by Patient

| Patient | 1 | 2 | 3 | 4 | 5 | 6 | 7 | 8 |
|---|---|---|---|---|---|---|---|---|
| Time from intubation to initiation | 3 | 3 | 3 | 3 | 3 | 1 | 1 | 1 |
| of OGT placement (min.) | | | | | | | | |
| Blood present in the oropharynx | N | N | Y | N | N | N | N | Y |
| prior to OGT placement? | | | | | | | | |
| Blood present in the oropharynx | N | N | Y | N | N | N | N | Y |
| after to OGT placement? | | | | | | | | |
| Number of attempts required to | 2 | 2 | 1 | 1 | 1 | 1 | 1 | 1 |
| place OGT | | | | | | | | |
| Number of practitioners involved | 2 | 1 | 1 | 1 | 1 | 1 | 1 | 1 |
| in OGT placement | | | | | | | | |
| Time from initiation to completion | 5 | 1 | 1 | 1 | 0.5 | 1 | 1 | 1 |
| of OGT placement (min.) | | | | | | | | |
| CXR to confirm ETT placement | N | N | N | N | N | N | N | N |
| prior to OGT placement? | | | | | | | | |
| Additional complications? | N | N | N | N | N | N | N | N |

Abbreviations: CXR = Chest X-ray;
ETT = Endotracheal tube;
OGT = Orogastric tube;
N = No;
Y = Yes

TABLE 1B
Study Results by Patient

| Patient | 9 | 10 | 11 | 12 | 13 | 14 | 15 | 16 |
|---|---|---|---|---|---|---|---|---|
| Time from intubation to | 2 | 1 | 1 | 24 h | 1 | 30 | 5 | 2 |
| initiation of OGT placement | | | | | | | | |
| (min.) | | | | | | | | |
| Blood present in the | N | N | N | Y | N | N | N | N |
| oropharynx prior to OGT | | | | | | | | |
| placement? | | | | | | | | |
| Blood present in the | N | N | N | Y | N | N | N | N |
| oropharynx after to OGT | | | | | | | | |
| placement? | | | | | | | | |
| Number of attempts required | 1 | 2 | 1 | 1 | 2 | 1 | 1 | 1 |
| to place OGT | | | | | | | | |
| Number of practitioners | 1 | 1 | 1 | 1 | 1 | 1 | 1 | 1 |
| involved in OGT placement | | | | | | | | |
| Time from initiation to | 0.25 | 1 | 1 | 1 | 1 | 1 | 1.5 | 1 |
| completion of OGT | | | | | | | | |
| placement (min.) | | | | | | | | |
| CXR to confirm ETT | N | N | N | Y | N | N | N | N |
| placement prior to OGT | | | | | | | | |
| placement? | | | | | | | | |
| Additional complications? | N | N | N | N | N | N | N | N |

Abbreviations: CXR = Chest X-ray;
ETT = Endotracheal tube;
OGT = Orogastric tube;
N = No;
Y = Yes

### Discussion

Oral endotracheal intubation using the GlideScope has become more popular for oral intubation in emergency departments and the intensive care unit, especially for difficult airways. orogastric tube placement immediately after intubation by providers performing endotracheal intubation is safe and provides real-time additional orogastric tube placement verification by direct visualization. It also reduces the time between oral endotracheal intubation and orogastric tube placement. The procedure was performed in a short period and needed few staff members, potentially reducing complications, and negating the need for additional radiograph and radiation exposure.

We hope that this study leads to larger studies in multiple centers with different providers and protocols. This would allow further testing of our technique. The use of this technique may change the approach used in critical care medicine for placement of orogastric tube and lead to the addition of the skill of orogastric tube placement to the skill set of the physician similar to the nursing staff. Limited studies have been published regarding the role of the physician and orogastric tube placement at the time of intubation, which is considered a core skill for emergency department and intensive care unit nurses. In our study, there was a very short learning curve for orogastric tube placement, and the physician and physician assistant were able to master this skill quickly.

Video visualization and verification of the orogastric tube passing through the hypopharyngeal esophagus reliably provides additional confirmation of accurate orogastric tube placement. It is the first and only method that is real-time and proactive. Because of the ease and convenience of this method, safety and practicality, it should be explored further on a larger scale at larger academic centers. This technique should be used more frequently by physicians performing intubations as a proactive technique for real-time confirmation of accurate orogastric tube placement. It may reduce complications, save time for the nursing staff, and reduce radiation exposure and cost.

1. A method of accurately placing an orogastric tube in a subject in need thereof, the method comprising:
inserting a distal portion of an airway visualization system into a hypopharynx of the subject; and
inserting an orogastric tube into a superior esophageal orifice of the subject.

2. The method of claim 1, wherein the airway visualization system is a fiber optic video-based system.

3. The method of claim 2, wherein the fiber optic video-based system is a GlideScope.

4. The method of claim 1, wherein the method does not include verifying accurate orogastric tube placement by radiography.

5. The method of claim 1 further comprising administering a paralytic agent to the subject before the step of inserting the orogastric tube.

6. The method of claim 1, wherein the subject has an endotracheal tube installed in a trachea of the subject, and wherein the method further comprises sweeping the endotracheal tube under a blade associated with the distal portion of the airway visualization system before the step of inserting the orogastric tube.

7. (canceled)

8. The method of claim 1 further comprising removing the distal portion of the airway visualization system from the hypopharynx of the subject after the step of inserting the orogastric tube.

9. The method of claim 1, wherein the step of inserting the orogastric tube comprises:
exposing the superior esophageal orifice with the airway visualization system; and
thereafter advancing a distal end of the orogastric tube posterior to the larynx at a midline of the subject and into the superior esophageal orifice.

10. The method of claim 1 further comprising injecting air into the orogastric tube after the step of inserting the orogastric tube, and auscultating an epigastric area of the subject to confirm placement of a distal end of the orogastric tube in a stomach of the subject.

11. The method of claim 1, wherein the method is associated with a substantially reduced risk of improper orogastric tube placement.

12. A method of accurately inserting a nasogastric tube in a subject in need thereof, the method comprising:
inserting a distal portion of an airway visualization system into a hypopharynx of the subject;
inserting a distal end of a nasogastric tube into a nostril of the subject;
advancing the nasogastric tube until the distal end is proximal to a superior esophageal orifice of the subject; and
advancing the distal end of the nasogastric tube into the superior esophageal orifice of the subject.

13. The method of claim 12, wherein the airway visualization system is a fiber optic video-based system.

14. The method of claim 13, wherein the fiber optic video-based system is a GlideScope.

15. The method of claim 12, wherein the method does not include verifying proper nasogastric tube placement by radiography.

16. The method of claim 12 further comprising administering a paralytic agent to the subject before the step of inserting the nasogastric tube.

17. The method of claim 12, wherein the subject has an endotracheal tube installed in a trachea of the subject, and wherein the method further comprises sweeping the endotracheal tube under a blade associated with the distal portion of the airway visualization system before the step of inserting the nasogastric tube.

18. (canceled)

19. The method of claim 12 further comprising removing the distal portion of the airway visualization system from the hypopharynx of the subject after the step of inserting the nasogastric tube.

20. The method of claim 12, wherein the step of inserting the nasogastric tube comprises:
exposing the superior esophageal orifice with the airway visualization system; and
thereafter advancing a distal end of the nasogastric tube posterior to the larynx at a midline of the subject and into the superior esophageal orifice.

21. The method of claim 12 further comprising injecting air into the nasogastric tube after the step of inserting the nasogastric tube, and auscultating an epigastric area of the subject to confirm placement of a distal end of the nasogastric tube in a stomach of the subject.

22. The method of claim 12, wherein the method is associated with a substantially reduced risk of improper nasogastric tube placement.

23-38. (canceled)
